(19) United States
(12) Patent Application Publication
(10) Pub. No.: US 2018/0235841 A1
(43) Pub. Date: Aug. 23, 2018

(54) WITHDRAWAL DEVICE FOR FLUIDS

(71) Applicant: Coltène/Whaledent GmbH & Co. KG, Langenau (DE)

(72) Inventors: Barbara Müller, Langenau (DE); Martin Schlüter, Wangen (DE); Johannes Wolfert, Langweid (DE); Werner Mannschedel, Langenau (DE)

(21) Appl. No.: 15/556,236

(22) PCT Filed: Mar. 1, 2016
(86) PCT No.: PCT/EP2016/054322

§ 371 Date: Sep. 6, 2017

(30) Foreign Application Priority Data
Mar. 6, 2015 (EP) 15000671.6

(51) Int. Cl. A61J 1/20; A61M 5/178; A61J 1/14; A61C 5/50; A61M 39/22; B65D 51/16; B65D 47/06

(52) U.S. Cl. CPC A61J 1/2096; A61M 5/1782; A61J 1/1418; A61M 2039/1083; A61M 39/22; B65D 51/1605; B65D 47/06; A61C 5/50

(57) ABSTRACT

A device for withdrawal of a fluid from a storage container has a body (11) which is configured so that it can be connected to an opening of the storage container, a rimmed cover (12) mounted on the upper end of the body (11), a connector (13) arranged laterally adjacent to the rim of the cover (12) for connecting the device to a syringe, and a suction line (16) which at its lower end leads vertically into the storage container and at its upper end opens into the connector (13).

The present invention relates to a device for withdrawing fluids from a storage container, especially in the field of medicine.

Devices for withdrawing fluids from a storage container play an important role, for example, in the field of medicine, especially in the field of dentistry, especially for fluids with which syringes are to be filled or loaded. The person skilled in the art often refers to the storage containers simply as bottles, injection bottles, phials or vials. The withdrawal device makes it possible to withdraw portions of the fluid stored in the storage container especially by means of a syringe.

DE 10 2013 012 353 A1 and DE 20 2012 007 582 U1 each describe a system for withdrawing medical fluids from a container. The described systems have a stopper which is inserted into the neck of the container and which, in the direction towards the exterior of the container, is provided in the centre with a female Luer connector for connection to a syringe having a male Luer connector and with an air-discharge opening between the centre and the rim of the stopper.

According to the teaching of DE 20 2012 007 582 U1, the female Luer connector is a Luer lock connector, and opposite the female Luer lock connector in the direction towards the interior of the container the stopper is provided with a suction tube which communicates with the female Luer lock connector via an opening in the stopper.

According to the teaching of DE 10 2013 012 353 A1, the female Luer connector is a Luer lock connector recessed into the stopper in such a way that the surface of the Luer lock connector terminates flush with the surface of the stopper, and opposite the female Luer lock connector in the direction towards the interior of the container the stopper is provided with a suction tube which communicates with the female Luer lock connector via a through-opening in the stopper, the suction tube having a through-opening the opening width of which is equal to the opening width of the through-opening in the stopper.

U.S. Pat. No. 8,671,994 B2 describes a device for filling injection syringes having a fluid conduit which connects an outlet located at the lower end of a storage vessel to a connector for injection syringes.

Those and further known solutions are associated with various disadvantages. Some solutions require that the bottle stands on its head or needs to be placed on its head for withdrawal, with the risk that fluid will run out of the bottle in an undesired way. In the case of some known solutions it is not possible to carry out genuinely one-handed fluid withdrawal for which the doctor or dentist or his assistant genuinely needs only one hand to attach a syringe and thus withdraw fluid from the bottle, without fluid being lost as a result of undesired spillage or running back into the bottle, with the associated risk that the fluid stored in the bottle will be contaminated.

In the case of some known solutions, valves must be operated by hand, and/or the bottle or an arrangement of several bottles (that is to say what is known as a station or loading station) must be held in place by hand. In the case of those or other known solutions a large number of small parts or operating elements increases the risk of user error and makes the arrangement more difficult to clean.

If there is an especially exposed Luer lock connector for injection syringes, such a connector usually has relatively large holes. As a result there is a risk of spillage. Therefore a cover must always be screwed on after use. There is also a risk that dirt will enter the bottle through the openings.

As a result of the complex and complicated set-up operation, which consists of a plurality of steps, known stations are frequently set up incorrectly. In addition, escapes or spillages of fluid can easily occur when the bottle is replenished. In such arrangements, after the set-up operation the fluid flows into a storage container of the station, for which reason the station, which especially has tubes and connections, always contains fluid. The risk of fluid escaping is very high here, and experience shows that many stations become leaky after a certain period of time.

Since the plastics material of the individual components of the station remains in contact with the fluid over prolonged periods of time, there is an increased risk that the particular fluid used, which is often chemically aggressive, will attack the plastics material until it totally fails. Individual parts of such bottles or stations cannot usually be replaced without requiring laborious repair.

The reservoir of such stations frequently cannot be cleaned or can be cleaned only with a large amount of effort. For that reason, contaminants can readily accumulate here.

The problem of the present invention is to define a novel device for withdrawal of fluids from a storage container that is as far as possible improved in comparison with the prior art. In particular, in respect of dental technology it is also a problem of the invention to define a device for withdrawal of fluids, especially irrigation solutions, for example a sodium hypochloride solution for irrigating root canal cavities.

That problem is solved by a subject matter according to the independent patent claim. Advantageous developments of the invention will be apparent from the dependent claims. The person skilled in the art can also combine features or groups of features of some embodiments with features or groups of features of other embodiments to form further, likewise advantageous embodiments of this invention not expressly described herein.

According to the invention there is provided a device for withdrawal of a fluid from a storage container, which device has a body which is configured so that it can be connected to an opening of the storage container. A rimmed cover is mounted on an upper end of the body. Laterally adjacent to the rim of the cover there is arranged a connector for connecting the device to a syringe. A suction line, which at its lower end leads vertically into the storage container, opens at its upper end into the connector.

Such a device is especially suitable for one-handed operation. It is very simple and intuitive to set up.

The body of the device is preferably configured so that it can be releasably connected, especially preferably by means of force-based engagement, to an opening of the storage container. According to a preferred embodiment, the body can be locked in place on the device. As a result it can be ensured that the device is always optimally aligned in the holder.

In a preferred embodiment of the invention, the body is at least in some regions hollow. In its interior it has a structure which tapers, preferably constantly and/or in stages, towards its lower end and is especially at least in some regions conical, through which structure the suction line leads into the storage container. Preferably the body is implemented so that the cover cannot be removed; the cover can therefore be attached to the body, for example, by adhesive bonding.

The suction line is preferably implemented as a suction tube.

Preferably the suction tube or the suction line closes the lower end of the conical structure air-tightly and/or fluid-tightly, especially preferably as a result of the external diameter of the suction tube fitting exactly into the internal diameter of the conical structure at the lower end thereof. In this way unintentional spillage or contamination of the fluid is counteracted.

In a further preferred embodiment of the invention, the body is configured so that it can be releasably connected to the opening of the storage container and preferably placed onto that opening from above or screwed onto that opening with the aid of a thread provided in the interior of the body. It is thereby possible for the body to be easily exchanged, for example in the event of contamination.

In a further preferred embodiment of the invention, the body has in its interior at least one cavity which is configured so that air is able to flow through that cavity into or out of the storage container at least when there is a pressure difference between the interior of the storage container and the exterior space. The body can also have two or more such cavities. Such forms of the device according to the invention promote ventilation of the storage container and counteract the formation of an undesirable pressure difference which could result in fluid flowing in an undesired way through the suction tube or through the suction line into or out of the storage container. According to a preferred embodiment, the cavities are formed in such a way that it is not possible for large amounts of fluid to spill out.

In a further preferred embodiment of the invention, the connector is configured preferably as a female Luer connector, especially preferably as a Luer lock connector, for connection to a syringe having preferably a male Luer connector or Luer lock connector. Such forms of the device according to the invention enable commercially available and sector-standard injection syringes and/or tube connections to be reliably connected to the connector of the device according to the invention. Alternatively, the connector is configured as a valve, for example as a Luer lock valve. As a result, after the syringe has been drawn up for the first time there is no longer any air in the suction line, so long as—even only a small amount of—fluid is present in the storage container. Further syringes can thus be filled without drawing up a greater amount of air into the syringe.

In a further preferred embodiment of the invention, the body of the device has a projection on its side opposite the connector. The projection is preferably configured in such a way that it is able to engage in and/or snap into a recess of a holder. Such forms of the device according to the invention facilitate its integration into a holder, especially into a table-mounted holder or a wall-mounted holder, with the result that a certain amount of stability can be imparted to the device, which is beneficial for easy and reliable one-handed operation. In addition, unintentional release of the device and/or of the storage container from the holder can thus be made more difficult.

In a further preferred embodiment of the invention, the cover has an opening for admitting air into or discharging air from the device. In this way, sufficient ventilation of the device and the storage container can be ensured even when the cover is closed. Optionally a filter can be provided in the opening or at some other location between the opening and the storage container in order to prevent the fluid in the storage container from being contaminated by contaminated inflowing air etc. According to a further embodiment, the body is not fully air-tight, so that no special ventilation opening is necessary.

According to the invention there is further provided a loading station which has a storage container, a holder and a withdrawal device according to one of the embodiments of the invention. The holder is preferably implemented in the form of a table-mounted holder and especially preferably with a base plate on which there is preferably arranged a vertical or upright structure having a recess in(to) which the projection of the withdrawal device according to the invention can engage and/or snap.

In a preferred embodiment of the loading station according to the invention, the projection on the side of the body opposite the connector is configured or arranged in such a way that it engages in or snaps into a recess in the holder in such a way that unintentional release of the device from the holder is made more difficult or is practically impossible.

According to the invention there is further provided a use of a product according to one of the described embodiments of the invention for withdrawal of an irrigation fluid for irrigating a root canal. The features of the withdrawal device according to the invention and its preferred embodiments contribute in a particular way to being able to meet the special requirements in respect of ease of operation, especially the as far as possible one-handed withdrawal of fluid and also the cleaning of the device, and in respect of adherence to hygiene regulations such as are typical for a dental practice.

The invention is explained in greater detail below on the basis of preferred exemplary embodiments and with reference to the accompanying Figures.

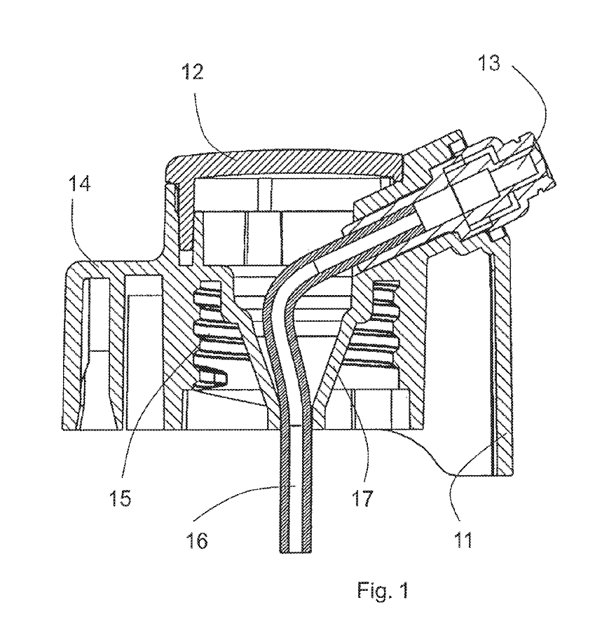

FIG. 1 shows a diagrammatic cross-section through an exemplary embodiment of a device according to the invention for withdrawal of fluids from a storage container;

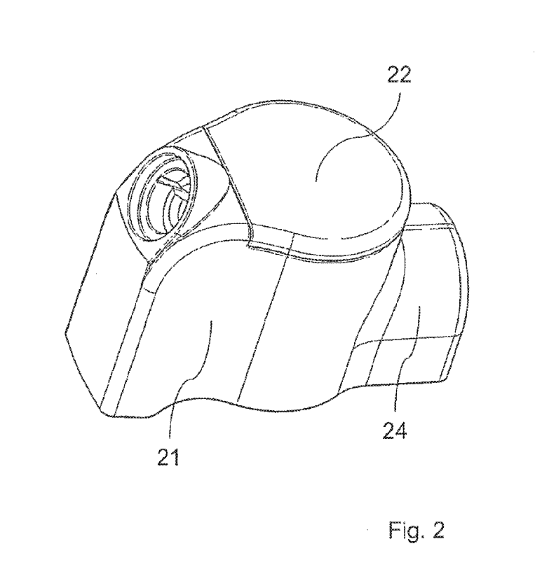

FIG. 2 shows a diagrammatic perspective view of an exemplary embodiment of a device according to the invention for withdrawal of fluids from a storage container;

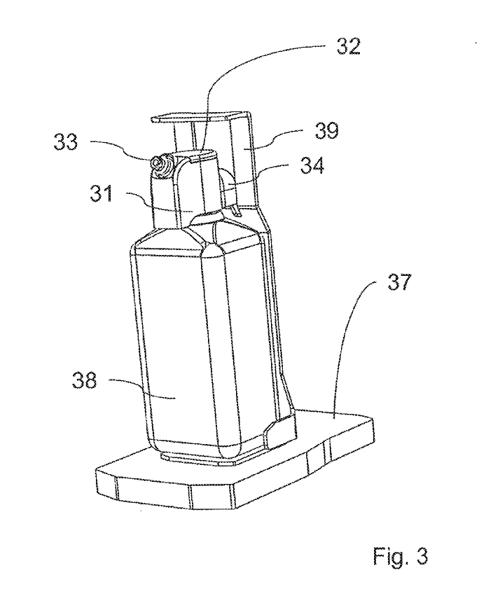

FIG. 3 shows a diagrammatic perspective view of an exemplary embodiment of a loading station with exemplary embodiments of a storage container and of a device according to the invention for withdrawal of fluids from a storage container.

FIG. 1 shows a diagrammatic cross-section through an exemplary embodiment of a device according to the invention for withdrawal of fluids from a storage container (not shown in FIG. 1). The device has a body 11 which is configured so that it can be connected to an opening of the storage container, a rimmed cover 12 mounted on the upper end of the body 11, a connector 13 arranged laterally adjacent to the rim of the cover 12 for connecting the device to a syringe, and a suction line 16 which at its lower end leads vertically into the storage container and at its upper end opens into the connector 13. The body 11 is at least partly hollow and has in its interior a structure 17 which tapers towards its lower end and is at least in some regions conical, through which structure the suction line 16 leads into the storage container. The body 11 of the device has a projection 14 on its side opposite the connector 13.

FIG. 2 shows a diagrammatic perspective view of an exemplary embodiment of a device according to the invention for withdrawal of fluids from a storage container (not shown in FIG. 2). The device has a body 21 which is configured so that it can be connected to an opening of the storage container, a rimmed cover 22 mounted on the upper end of the body 21, and a fitting arranged laterally adjacent to the rim of the cover 22 for a connector (not shown) for connecting the device to a syringe. The body 21 of the device has a projection 24 on its side opposite the fitting for a connector.

FIG. 3 shows a diagrammatic perspective view of an exemplary embodiment of a loading station with exemplary embodiments of a storage container 38 and of a device 31, 32, 33, 34 according to the invention for withdrawal of fluids from that storage container 38. The storage container 38 stands on a base plate 37 of a holder 37, 39, which has a vertical or upright structure 39. The projection 34 of the withdrawal device engages in a recess of the vertical structure 39 or is snapped into that recess. FIG. 3, on account of the perspective chosen, does not show that the structure 39 can additionally or alternatively have a projection which engages in a recess corresponding to the recess 18 in FIG. 1.

The following reference signs have been used in the Figures:
11 withdrawal device
12 cover
13 connector
14 projection
15 thread
16 suction line, suction tube
17 conical structure
18 recess
21 withdrawal device
22 cover
24 projection
31 withdrawal device
32 cover
33 connector
34 projection
37 base plate
38 storage container
39 holder

1-11. (canceled)

12. A device for withdrawal of a fluid from a storage container, the device having:
a) a body which is configured so that it can be connected to an opening of the storage container;
b) a rimmed cover mounted on the upper end of the body;
c) a connector arranged laterally adjacent to the rim of the cover for connecting the device to a syringe, and
d) a suction line which at its lower end leads vertically into the storage container and at its upper end opens into the connector.

13. A device of claim 12 wherein the body is at least partly hollow and has in its interior a structure which tapers towards its lower end and is especially at least in some regions conical, through which structure the suction line leads into the storage container.

14. A device of claim 12 wherein the body is configured so that it can be releasably connected to the opening of the storage container, preferably placed onto that opening from above or screwed onto that opening with the aid of a thread provided in the interior of the body and/or optionally locked in place on the storage container.

15. A device of claim 12 wherein the body has in its interior at least one cavity which is configured so that air is able to flow through that cavity into or out of the storage container at least when there is a pressure difference between the interior of the storage container and the exterior space.

16. A device of claim 12 wherein the connector is configured for connection to a syringe.

17. A device of claim 12 wherein the connector is configured as a female Luer connector for connection to a syringe having a male Luer connector.

18. A device of claim 12 wherein the connector is configured as a female Luer lock connector for connection to a syringe having a male Luer lock connector.

19. A device of claim 12 wherein the connector is configured as a valve for connection to a syringe.

20. A device of claim 12 wherein the connector is configured as a female Luer valve for connection to a syringe having a male Luer connector.

21. A device of claim 12 wherein the connector is configured as a female Luer lock valve for connection to a syringe having a male Luer lock connector.

22. A device of claim 12 wherein the body of the device has a projection on its side opposite the connector.

23. A device of claim 12 wherein the device comprises an opening for admitting air into or discharging air from the device.

24. A loading station comprising a storage container, a holder and a withdrawal device of claim 1.

25. A loading station of claim 24 wherein the projection on the side of the body opposite the connector engages in or snaps into a recess in the holder in such a way that unintentional release of the device from the holder is made more difficult.

26. A method for withdrawing an irrigation fluid for irrigating a root canal, comprising using a device of claim 12.
